(19) United States
(12) Patent Application Publication
(10) Pub. No.: US 2007/0248669 A1
(43) Pub. Date: Oct. 25, 2007

(54) Oral sustained release formulation

(76) Inventors: Fang-Hsiung Hsiao, Tainan Hsien (TW); Ya-Ching Changchien, Kaohsiung (TW)

Correspondence Address: ANDREWS KURTH LLP, 1350 I STREET, N.W., SUITE 1100, WASHINGTON, DC, 20005, US

(21) Appl. No.: 11/410,017

(22) Filed: Apr. 25, 2006

(51) Int. Cl. A61K 31/137; A61K 9/24

(52) U.S. Cl. 424/471; 514/649

(57) ABSTRACT

The present invention relates to an oral sustained release formulation comprising a core, a medicinal layer containing vanlafaxine or a pharmaceutically acceptable salt of venlafaxine and a release-modulating layer containing a release-modulating agent. The present invention also relates to a method for preparing an oral sustained release formulation, and to a pharmaceutical composition containing the oral sustained release formulation prepared by the method.

### BACKGROUND OF THE INVENTION

1. Field of the Invention

The present invention relates to a pharmaceutical composition of an oral sustained release formulation containing venlafaxine or a pharmaceutically acceptable salt of venlafaxine.

2. Description of the Related Art

Venlafaxine, 1-[2-(dimethylamino)-1-(4-methoxyphenyl)-ethyl]cyclohexanol, is an important drug in the neuropharmacological field and is used for the treatment of depression. Venlafaxine hydrochloride was previously administered to adult patients in compressed tablet form in doses ranging from 75 to 375 mg/day, in separate doses two or three times a day. In therapeutic dosing with venlafaxine hydrochloride tablets, rapid dissolution results in a rapid increase in blood plasma levels shortly after administration of the active compound. A decrease in blood plasma levels follows over several hours as the active compound is eliminated or metabolized until normal plasma levels are approached about twelve hours after administration, which requires additional dosing with the drug. With the daily multiple dosing regimen, the most common side effect is nausea, experienced by about forty five percent of patients under treatment with venlafaxine hydrochloride, which results in vomiting in about seventeen percent of the patients.

Extended release formulations of venlafaxine maintain the plasma level of the active compound with an effect better than instant release formulations and reduce the probability of side effects. Extended release formulations are conventionally produced as compressed tablets by hydrogel tablet technology. To produce these sustained release tablets, the active ingredient is conventionally mixed with cellulose ethers such as methylcellulose, ethyl cellulose or hydroxypropylmethylcellulose, with or without other excipients, and the resulting mixture is pressed into tablets. When the tablets are orally administered, the cellulose ethers in the tablets swell upon hydration from moisture in the digestive system, thereby limiting exposure of the active ingredient to moisture. As the cellulose ethers are gradually leached away by moisture, water penetrates deeper and deeper into the gel matrix, and the active ingredient slowly dissolves and diffuses through the gel, making it available for sustained release.

TW Pat. Publication No. 493,993 discloses an extended release formulation of venlafaxine hydrochloride. The formulation comprises a capsule containing spheroids composed of from about 30% to about 40% venlafaxine hydrochloride by weight, about 50% to about 70% microcrystalline cellulose by weight and from about 0.25% to about 1% by weight of hydroxypropylmethylcellulose. The spheroids are coated with a film coating composition composed of about 80% to about 90% ethyl cellulose and about 10% to about 20% hydroxypropylmethylcellulose.

TW Pat. Publication No. 555,568 discloses an encapsulated, extended release formulation of venlafaxine hydrochloride that comprises a capsule containing spheroids with a therapeutically effective amount of venlafaxine hydrochloride in the core. The core of the spheroid does not contain any hydroxypropylmethylcellulose. The core of the spheroid comprises about 30% to about 40% venlafaxine hydrochloride and about 60% to about 70% by weight microcrystalline cellulose and is coated with a mixture of about 80% to about 90% by weight ethyl cellulose and about 10% to about 20% by weight hydroxypropylmethylcellulose.

The methods for providing the above two formulations include extruding the mixtures containing venlafaxine hydrochloride and cellulose, followed by spheronization. However, the process for preparing the core of the spheroids requires several steps and is time-consuming and costly. In addition, the spheroids produced are irregular and have great variation in size.

In view of the existing shortcomings in the prior art, a need still exists for an oral sustained release formulation of venlafaxine to obviate or mitigate the aforementioned problems.

### SUMMARY OF THE INVENTION

The present invention provides an oral sustained release formulation of venlafaxine or a pharmaceutically acceptable salt of venlafaxine, a method to prepare said oral sustained release formulation and a pharmaceutical composition comprising said sustained release formulation.

The oral sustained release formulation in accordance with the present invention comprises a core, a medicinal layer and a release-modulating layer.

The core is a matrix. The medicinal layer contains vanlafaxine or a pharmaceutically acceptable salt of vanlafaxine and coats the core. The release-modulating layer provides the desired level of dissolution, contains a release-modulating agent and coats the medicinal layer.

Preferably, the core the core is made by one or more excipients.

More preferably, the core is selected from a group consisting of sugar granule, sugar-starch granule and microcrystal cellulose.

More preferably, the excipient is one or more selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

Preferably, the medicinal layer further comprises about 20% to 35% (w/w) venlafaxine or venlafaxine hydrochloride.

Preferably, the medicinal layer further comprises a binder, a plastic agent, an anti-binding agent and/or a diluent.

More preferably, the binder in the medicinal layer is one or more selected from the group of polyvinyl pyrrolidone (PVP), hydroxypropyl cellulose (HPC), hydroxypropyl methylcellulose (HPMC), vinyl acetate (VA), polyvinyl alcohol (PVA), methylcellulose (MC), ethyl cellulose (EC), hydroxypropyl methylcellulose phthalate (HPMCP), cellulose acetate propionate (CAP), xanthan gum, alginic acid, alginate, methacrylic acid-methacrylate copolymer, methacrylic acid-methyl methacrylate copolymer and polyvinyl acetate phthalate (PVAP).

More preferably, the binder is ethyl cellulose.

Preferably, the medicinal layer comprises about 3% to 10% (w/w) ethyl cellulose as the binder.

Preferably, the plastic agent in the medicinal layer is one or more selected from the group consisting of glycerol, polyethylene glycol, triethyl citrate, tributyl citrate, 3-propyl acetate, diethyl phthalate and dibutyl phthalate.

Preferably, the diluent in the medicinal layer is one or more selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

Preferably, the release-modulating agent is one or more selected from the group consisting of polyvinyl pyrrolidone (PVP), HPMC, EC, methacrylic acid-methacrylate copolymer and methacrylic acid-methyl methacrylate copolymer.

Preferably, the release-modulating layer further comprises an anti-binding agent and/or a plastic agent.

Preferably, the anti-binding agent in the release-modulating layer is one or more selected from the group consisting of talcum powder, titanium dioxide, stearic acid, stearic salt, sodium stearyl fumarate, glyceryl behenate, kaolin and aerosol.

Preferably, the plastic agent in the release-modulating layer is one or more selected from the group consisting of glycerol, polyethylene glycol, triethyl citrate, tributyl citrate, 3-propyl acetate, diethyl phthalate and dibutyl phthalate.

Preferably, the plastic agent is polyethylene glycol or triethyl citrate.

Preferably, the release-modulating layer comprises about 80% to 90% (w/w) ethyl cellulose and about 10% to 20% (w/w) dibutyl phthalate.

Preferably, the sustained release formulation comprises about 30% to 40% (w/w) the core, about 20% to about 35% (w/w) venlafaxine or venlafaxine hydrochloride and about 5% to about 20% (w/w) release-modulating layer.

More preferably, the sustained release formulation comprises about 35% (w/w) the core, about 30% (w/w) venlafaxine or venlafaxine hydrochloride and about 10% to about 16% (w/w) release-modulating layer.

The method to prepare said sustained release formulation comprises (a) providing a core, (b) coating one or more medicinal layers containing venlafaxine or a pharmaceutically acceptable salt of venlafaxine on the core to obtain a granule, (c) coating one or more release-modulating layers containing a release-modulating agent on the granule obtained from step (b), and optionally (d) repeating step (b) and step (c).

Preferably, the method further comprises repeating (b) and (c) once or more.

Preferably, the core is provided by wet granulation.

Preferably, the core is of a diameter of from 0.5 to 0.85 mm.

Preferably, the medicinal layer is sprayed around the core.

Preferably, the release-modulating layer is sprayed around the medicinal layer.

Yet another aspect of the present invention is related to a pharmaceutical composition, which that comprises' the sustained release formulation of venlafaxine or a pharmaceutically acceptable salt of venlafaxine made by the method according to the present invention.

Other aspects, advantages and novel features of the invention will become more apparent from the following detailed description when taken in conjunction with the accompanying drawings.

### BRIEF DESCRIPTION OF THE DRAWINGS

FIG. 1 is a cross-section view of a sustained release formulation of venlafaxine in accordance with the present invention;

FIG. 2 is a graph of dissolution profiles of a sustained release formulations of venlafaxine in accordance with the present invention in Example 2 and a control;

FIG. 3 is a graph of dissolution profiles of a sustained release formulation of venlafaxine in accordance with the present invention in Example 3 and a control; and

FIG. 4 is a graph of dissolution profiles of a sustained release formulation of venlafaxine in accordance with the present invention in Example 4 and a control.

### DETAILED DESCRIPTION OF THE PREFERRED EMBODIMENTS

The present invention provides an oral sustained release formulation of venlafaxine or a pharmaceutically acceptable salt of venlafaxine, a method to prepare said oral sustained release formulation and a pharmaceutical composition comprising said sustained release formulation.

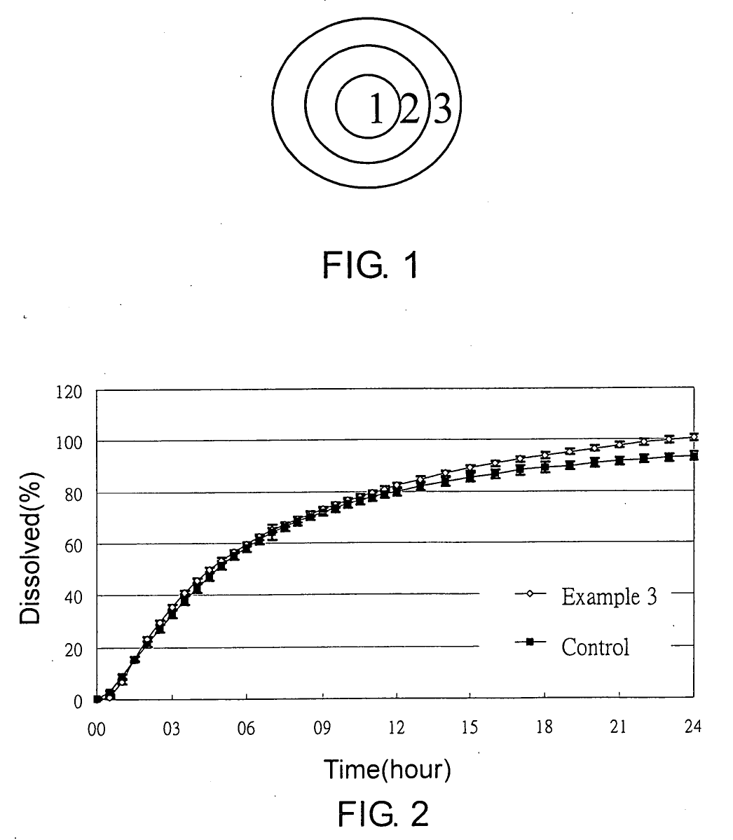

With reference to FIG. 1, the oral sustained release formulation of venlafaxine or a pharmaceutically acceptable salt of venlafaxine can make up an oral dosage unit alone or be mixed with excipients to form granules or tablets or be held in capsules, is produced using particle-encapsulating technology and comprises a core (1), a medicinal layer (2) and a release-modulating layer (3), and venlafaxine is 1-[2-(dimethylamino)-1-(4-methoxyphenyl)ethyl]cyclohexanol.

Before describing the oral sustained release formulation, definitions of a number of key terms are provided to avoid possible ambiguity.

The term “sustained release” as used herein refers to formulation or dosage units that are slowly and continuously dissolved and absorbed in the stomach and gastrointestinal tract over a period of time. The term refers to the release of an active ingredient at such a rate that blood levels are maintained within a therapeutic range but below toxic levels over an extended period of time, e.g., 4 to 24 hours or even longer.

The term “procedures described in Examples” refers to the way to mix compounds and produce the oral sustained release formulation of venlafaxine or a pharmaceutically acceptable salt of venlafaxine.

The term “composition” as used herein indicates more than one active pharmaceutical ingredient.

The term “formulation” as used herein refers to any form commonly used for pharmaceutical administration, including solids, liquids, suspensions, creams and gels. For purposes of the present invention, the formulation is preferably a granule.

The core (1) does not have any medicinal effects, may be a granule, a tablet or microtablet, has a diameter, is a matrix, constitutes about 30% to 40% (w/w) of the oral sustained release formulation, more preferably constitutes about 35% of the oral sustained release formulation and contains one or more excipients. The matrix is selected from a group consisting of sugar granules, sugar and starch granules and microcrystal cellulose. The excipient is selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin or castor oil.

The medicinal layer (2) contains vanlafaxine or a pharmaceutically acceptable salt of vanlafaxine, preferably comprises about 20% to 35% (w/w) venlafaxine or venlafaxine hydrochloride, more preferably constitutes about 30% (w/w) venlafaxine or venlataxine hydrochloride, may further comprise a binder, a plastic agent, an anti-binding agent, a diluent or a combination of the foregoing and coats the core (1).

Preferably, the binder is one or more selected from the group consisting of polyvinyl pyrrolidone (PVP), gelatin, hydroethyl cellulose (HEC), hydroxypropyl cellulose (HPC), hydroxypropyl methylcellulose (HPMC), vinyl acetate (VA), polyvinyl alcohol (PVA), methylcellulose (MC), ethyl cellulose (EC), hydroxypropyl methylcellulose phthalate (HPMCP), cellulose acetate propionate (CAP), xanthan gum, alginic acid, alginate, methacrylic acid-methacrylate copolymer, methacrylic acid-methyl methacrylate copolymer and polyvinyl acetate phthalate (PVAP). More preferably, the binder is ethyl cellulose, and the medicinal layer comprises about 3% to 10% (w/w) ethyl cellulose.

The plastic agent is one or more selected from the group consisting of glycerol, polyethylene glycol, triethyl citrate, tributyl citrate, 3-propyl acetate, diethyl phthalate and dibutyl phthalate.

The anti-binding agent is one or more selected from the group consisting of talcum powder, titanium dioxide, stearic acid, stearic salt, sodium stearyl fumarate, glyceryl behenate, kaolin and aerosol.

The diluent is one or more selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

The release-modulating layer provides the desired level of dissolution, contains a release-modulating agent, preferably further comprises an anti-binding agent, a plastic agent or both, constitutes about 5% to about 20% (w/w) of the oral sustained release formulation, more preferably constitutes about 10% to about 16% (w/w) of the oral sustained release formulation and coats the medicinal layer.

The release-modulating agent is one or more selected from the group consisting of polyvinyl pyrrolidone (PVP), HPMC, EC, methacrylic acid-methacrylate copolymer and methacrylic acid-methyl methacrylate copolymer. Preferably, the release-modulating layer forms about 5 to about 20 percent on a weight/weight basis of the final product with best results obtained at from about 6 to about 8 percent (w/w).

The anti-binding agent is one or more selected from the group consisting of talcum powder, titanium dioxide, stearic acid, stearic salt, sodium stearyl fumarate, glyceryl behenate, kaolin and aerosol.

The plastic agent in the release-modulating layer is one or more selected from the group consisting of glycerol, polyethylene glycol, triethyl citrate, tributyl citrate, 3-propyl acetate, diethyl phthalate and dibutyl phthalate. Most preferably, the plastic agent in the release-modulating layer is polyethylene glycol, triethyl citrate or dibutyl phthalate.

More preferably, the release-modulating layer in the oral sustained release formulation comprises about 80% to 90% (w/w) ethyl cellulose and about 10% to 20% (w/w) dibutyl phthalate.

Specifically, the sustained release formulations according to this invention comprise from about 20 to about 35 percent venlafaxine or a pharmaceutically acceptable salt of venlafaxine. More specifically, the sustained release formulations according to this invention comprise from about 30 to about 40 percent neutral core, from about 3 to about 10 percent ethyl cellulose, and from about 5 to about 20 percent release-modulating layer, all on a weight/weight basis. Preferably, the spheroid formulations contain about 30 percent venlafaxine or a pharmaceutically acceptable salt of venlafaxine, about 35 percent neutral core, about 5 percent ethyl cellulose (Aquacoat® ECD30, which has a viscosity of N.M.T.150 cps for 30% aqueous solutions and a ethyl cellulose content of 24.5-29.5%), and from about 10 to 16 percent release-modulating layer. The release-modulating layer is comprised of about 80 to 90 percent of ethyl cellulose and about 10 to 20 percent dibutyl phthalate on a weight/weight basis. Preferably, ethyl cellulose has an ethoxy content of 44.0 to 51% and a viscosity of 50 cps for a 5% aqueous solution. In a preferred embodiment, ethyl cellulose may be Aquacoat® ECD30 ethyl cellulose.

The oral sustained release formulation according to the present invention contains a core, a medicinal layer coating around the core and a release-modulating layer coating around the medicinal layer.

The method to prepare said oral sustained release formulation comprises (a) providing a core (1), (b) coating one or more medicinal layers (2) containing venlafaxine or a pharmaceutically acceptable salt of venlafaxine on the core (1) to obtain a granule, (c) coating one or more release-modulating layers (3) containing a release-modulating agent on the granule obtained from step (b), and optionally (d) repeating step (b) and step (c).

In the step (a) providing a core (1), the core (1) may be made by one or more excipients or may obtain from a supplier, and the excipients may be mixed by wet granulation in a fluidized bed granulator to form the core (1). The preferred diameter of the core may be about 0.5 to 0.85 mm. The core may be a tablet or a microtablet, and may be formed by direct compression.

The excipients according to the present invention may be selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

The core that may be employed according to the present invention from suppliers. The core obtained from a supplier may be generally a sucrose-grained core, sucrose-starch grained core or microcrystalline cellulose grained core.

Step (b) coating one or more medicinal layers (2) containing venlafaxine or a pharmaceutically acceptable salt of venlafaxine on the core (1) to obtain a granule comprises mixing venlafaxine or a pharmaceutically acceptable salt of venlafaxine, an anti-binding agent and optionally a binder, a plastic agent or a diluent to obtain a solution that is applied to and coats the core (1). The solution may be sprayed onto the core with an airbrush to form a medicinal layer around the core after the solution dries.

The binder according to the present invention may be selected from the group consisting of polyvinyl pyrrolidone (PVP), gelatin, hydroethyl cellulose (HEC), hydroxypropyl cellulose (HPC), hydroxypropyl methylcellulose (HPMC), vinyl acetate (VA), polyvinyl alcohol (PVA), methylcellulose (MC), ethyl cellulose (EC), hydroxypropyl methylcellulose phthalate (HPMCP), cellulose acetate propionate (CAP), xanthan gum, alginic acid, alginate, methacrylic acid-methacrylate copolymer, methacrylic acid-methyl methacrylate copolymer and polyvinyl acetate phthalate (PVAP).

The plastic agent according to the present invention may be selected from the group consisting of glycerol, polyethylene glycol, triethyl citrate, tributyl citrate, 3-propyl acetate, diethyl phthalate and dibutyl phthalate.

The diluent according to the present invention may be selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

The anti-binding agent according to the present invention may be selected from the group consisting of talcum powder, titanium dioxide, stearic acid, stearic salt, sodium stearyl fumarate, glyceryl behenate, kaolin and aerosol.

In step (c), a dissolution-modulating agent, a plastic agent and an anti-binding agent are mixed in a solvent to form a release-modulating agent. The release-modulating agent may be sprayed onto the core coated with the medicinal layer with an airbrush and become a release-modulating layer after drying.

The plastic agent according to the present invention may be selected from the group consisting of glycerol, polyethylene glycol, triethyl citrate, tributyl citrate, 3-propyl acetate, diethyl phthalate and dibutyl phthalate.

The anti-binding agent according to the present invention may be selected from the group consisting of talcum powder, titanium dioxide, stearic acid, stearic salt, sodium stearyl fumarate, glyceryl behenate, kaolin and aerosol.

The solvent according to the present invention may be selected from the group consisting of water, alcohol, acetone, isopropanol and methylene chloride.

In a preferred embodiment, the method described in the present invention describes as follow.

A. A granule was provided as a core, the core may be made from an excipient that may be sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil. The core may be a commercial product, such as a sucrose-grained core, a sucrose-starch grained core or a microcrystalline cellulose grained core.

B. A medicinal layer containing active pharmaceutical ingredient may be coated around the core. The medicinal layer may comprise effective amount of active pharmaceutical ingredient and anti-binding agent, optionally, a binder, a plastic agent and/or a diluent may be added. The binder may be polyvinyl pyrrolidone (PVP), gelatin, hydroethyl cellulose (HEC), hydroxypropyl cellulose (HPC), hydroxypropyl methylcellulose (HPMC), vinyl acetate (VA), polyvinyl alcohol (PVA), methylcellulose (MC), ethyl cellulose (EC), hydroxypropyl methylcellulose phthalate (HPMCP), cellulose acetate propionate (CAP), xanthan gum, alginic acid, alginate, methacrylic acid-methacrylate copolymer, methacrylic acid-methyl methacrylate copolymer or polyvinyl acetate phthalate (PVAP). The diluent may be lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin or castor oil. The plastic agent may be glycerol, polyethylene glycol, triethyl citrate, tributyl citrate, 3-propyl acetate, diethyl phthalate or dibutyl phthalate. The anti-binding agent may be talcum powder, titanium dioxide, stearic acid, stearic salt, sodium stearyl fumarate, glyceryl behenate, kaolin or aerosol.

C. A release-modulating layer may be coated around the medicinal layer. The release-modulating layer may be any kinds of the conventional release-modulated compounds.

D. Selectively, the medicinal layer and the release-modulating layer may be coated layer by layer in turn.

Because venlafaxine or a pharmaceutically acceptable salt of venlafaxine is a high dissolution drug, the present invention is relates to a sustained release formulation which may comprise a core, a medicinal layer and a release-modulating layer. The method disclosed in the present invention may include, but not limited to, venlafaxine or a pharmaceutically acceptable salt of venlafaxine.

In a preferred embodiment, the oral sustained release formulation according to the present invention has a core (1), a medicinal layer (2) and a release-modulating layer (3). The core (1) may be a granule. The medicinal layer (2) coats the core (1). The release-modulating layer (3) is coats the medicinal layer (2).

The oral sustained release formulation according to the present invention may comprise more than one active pharmaceutical ingredient, such as fluoxetine, fluvoxamine, paroxetine or galantamine. Furthermore, the oral sustained release formulation according to the present invention may be formed as a granule, capsule or tablet.

The detail of the oral sustained release formulation made according to the present invention is described as follow.

(a) First, a pharmaceutical acceptable excipient was formed a core (1), the core (1) may be made by wet granulation. The core may be made by a fluidized bed granulator. In a preferred embodiment, the core (1) may have 3 to 5 mm diameter. If the oral sustained release formulation is a tablet or a microtablet, a preferred diameter of the core (1) may be 0.5 to 0.85 mm.

(b) Second, the active pharmaceutical ingredient, such as venlafaxine or venlafaxine hydrochloride salts, was dissolved in a solution or a suspension. Optionally, a binder, a plastic agent or diluents may be added into the solution or the suspension to form a medicinal solution. In step (b), the medicinal solution may be sprayed on the core (1) to form a medicinal layer (2). In a preferred embodiment, the amount of venlafaxine or a pharmaceutically acceptable salt thereof may be in a range of from about 20% to 35% (w/w), the plastic agent may be in a range of from about 0% to 10%, and the anti-binding agent may be in a range of from about 0.5% to 30% (w/w).

(c) Third, a release-modulating agent, a plastic agent, an anti-binding agent and solution may be prepared to form a release-modulating solution. The release-modulating solution may be sprayed on and dried the medicinal layer (2) containing the core (1) to form the release-modulating layer (3). The plastic agent may be the same or different from the plastic agent used in step (a). The anti-binding agent may be talcum powder, titanium dioxide, stearic acid, stearic salt, sodium stearyl fumarate, glyceryl behenate, kaolin or aerosol. The solution may be water, alcohol, acetone, isopropanol or methylene chloride.

In a preferred embodiment, the medicinal layer of the oral sustained release formulation may comprise about 34% (w/w) venlafaxine or venlafaxine hydrochloride, about 4% ethyl cellulose (EC) and about 0.8% triethyl citrate which are added into about 39.0% water to form the medicinal solution. The medicinal solution may be coated on the core to form the medicinal layer. Then the granules containing medicinal layer and the core may be sieved to obtain the granules with 0.85 μm (20 mesh) to 1.18 μm (16 mesh).

In a preferred embodiment, the release-modulating layer may be made by stirring 70.8% ethyl cellulose (Aquacoat® ECD30) and 14.2% (w/v) dibutyl phthalate to form the release-modulating solution and may be sprayed on the granules containing medicinal layer and the core.

In a preferred embodiment, the granules having the core, the medicinal layer and the release-modulating layer may be sieved to obtain the granules with 1.18 μm (116 mesh) and 1.40 μm (14 mesh). Then the obtained granules may be stored in a capsule.

Other features and advantages of the present invention will be apparent from the following description of the preferred embodiments and from the claims.

### EXAMPLES

The following examples illustrate various aspects of the present invention but do not limit the claims in any manner whatsoever.

### Example 1

### Oral Sustained Release Formulation (1)

Formulation:

| | | | |
|---|---|---|---|
| (a) | Core: | 270 | g |
| (b) | Medicinal layer: | | |
| | Venlafaxine hydrochloride | 280 | g |
| | Ethyl cellulose (Aquacoat ® ECD30) | 111.87 | g |
| | Triethyl citrate | 6.53 | g |
| | Titanium dioxide (TiO₂) | 9.9 | g |
| | Talcum powder | 66 | g |
| | Pure water | 210 | ml |
| (c) | Release-modulating layer: | | |
| | Ethyl cellulose (Aquacoat ® ECD30) | 155.8 | g |
| | Dibutyl phthalate | 9.35 | g |
| | Titanium dioxide (TiO₂) | 1.65 | g |
| | Talcum powder | 8.25 | g |
| | Pure water | 100 | ml |

Procedures:

(1) 111.87 g ethyl cellulose, 6.53 g triethyl citrate and 210 ml pure water were mixed to be a solution.

(2) 280 g venlafaxine hydrochloride was dissolved or suspended in the solution obtained from procedure (1).

(3) 9.9 g TiO2 and 66 g talcum powder were added to the solution obtained from procedure (2) to form a medicinal agent; the medicinal agent was sprayed on the core in a fluidized bed granulator, and a core with venlafaxine was obtained after spraying and drying.

(4) 155.8 g ethyl cellulose, 9.35 g dibutyl phthalate, 1.65 g TiO2, 8.25 g talcum powder and 100 ml pure water were mixed to be a release-modulating agent, and the release-modulating agent was sprayed onto the core with venlafaxine to obtain the oral sustained release formulation.

### Example 2

### Oral Sustained Release Formulation (2)

Formulation:

| | | | |
|---|---|---|---|
| (a) | Core: | 270 | g |
| (b) | Medicinal layer: | | |
| | Venlafaxine hydrochloride | 280 | g |
| | Tthyl cellulose (Aquacoat ® ECD30) | 111.87 | g |
| | Triethyl citrate | 6.53 | g |
| | Titanium dioxide (TiO2) | 9.9 | g |
| | Talcum powder | 66 | g |
| | Pure water | 210 | ml |
| (c) | Release-modulating layer: | | |
| | Methacrylic acid-methacrylate copolymer | 220 | g |
| | (Eudragit ® NE30D) | | |
| | Diethyl phthalate | 13.2 | g |
| | Talcum powder | 19.8 | g |
| | Pure water | 120 | ml |

Procedures:

(1) 111.87 g ethyl cellulose, 6.53 g triethyl citrate and 210 ml pure water were mixed to be a solution.

(2) 280 g venlafaxine hydrochloride was dissolved or suspended in the solution obtained from procedure (1).

(3) 9.9 g TiO₂ and 66 g talcum powder were added to the solution obtained from procedure (2) to form a medicinal agent; the medicinal agent was sprayed on the core in a fluidized bed granulator, and a core with venlafaxine was obtained after spraying and drying.

(4) 150 g methacrylic acid-methacrylate copolymer, 13.2 g diethyl phthalate, 19.8 g talcum powder and 120 ml pure water were mixed to be a release-modulating agent, and the release-modulating agent was sprayed onto the core with venlafaxine to obtain the oral sustained release formulation.

### Example 3

### Oral Sustained Release Formulation (3)

Formulation:

| | | | |
|---|---|---|---|
| (a) | Core: | 300 | g |
| (b) | Medicinal layer: | | |
| | Venlafaxine hydrochloride | 254 | g |
| | Methacrylic acid-methacrylate copolymer | 180.8 | g |
| | (Eudragit ® NE30D) | | |
| | Triethyl citrate | 10.8 | g |
| | Titanium dioxide (TiO₂) | 9.0 | g |
| | Talcum powder | 66 | g |
| | Pure water | 240 | ml |
| (c) | Release-modulating layer: | | |
| | Ethyl cellulose (Aquacoat ® ECD30) | 155.8 | g |
| | Dibutyl phthalate | 9.35 | g |
| | Titanium dioxide (TiO₂) | 1.65 | g |
| | Talcum powder | 8.25 | g |
| | Pure water | 100 | ml |

Procedures:

(1) 180.8 g methacrylic acid-methacrylate copolymer, 10.8 g triethyl citrate and 240 ml pure water were mixed to be a solution.

(2) 300 g venlafaxine hydrochloride was dissolved or suspended in the solution obtained from procedure (1).

(3) 9.9 g TiO₂ and 66 g talcum powder were added to the solution obtained from procedure (2) to form a medicinal agent; the medicinal agent was sprayed on the core in a fluidized bed granulator, and the core with venlafaxine was obtained after spraying and drying.

(4) 155.8 g ethyl cellulose, 9.35 g dibutyl phthalate, 1.65 g TiO₂, 8.25 g talcum powder and 100 ml pure water were mixed to be a release-modulating agent, and the release-modulating agent was sprayed onto the core with venlafaxine to obtain the oral sustained release formulation.

### Example 4

### Control Test

Experiments: Testing of the oral sustained release formulation made according to Examples 1-3 and a conventional venlafaxine hydrochloride capsule formulation:

The acceptability of the coating level of the release-modulating layer was determined by analysis of the dissolution rate of the granule stored inside the capsules. The dissolution procedure was determined by USP Apparatus 1 (Basket) at 100 rpm in 0.1 N hydrochloric acid at 37° C. When given granules with the release-modulating layer released the venlafaxine too slowly to comply with the desired dissolution rate, a portion of granules without/with a lower coating level of the release-modulating layer may be added and stored in the capsule to obtain a suitable dissolution rate. If the granules with the release-modulating layer may release drug too rapidly and an additional release-modulating layer may be coated on the granules to give the desired dissolution rate.

The sustained release formulations obtained from Examples 1 to 3 were respectively stored in capsules and tested. The venlafaxine doses for testing were respectively 37.5 mg, 75 mg and 100 mg. Dissolution rate of the capsules containing venlafaxine or a pharmaceutically acceptable salt of venlafaxine was determined by the method described in the U.S. Pharmacopoeia (USP) using apparatus 1 (Basket) at 100 rpm on 0.9 L of 0.1 N HCl_{(aq)}.

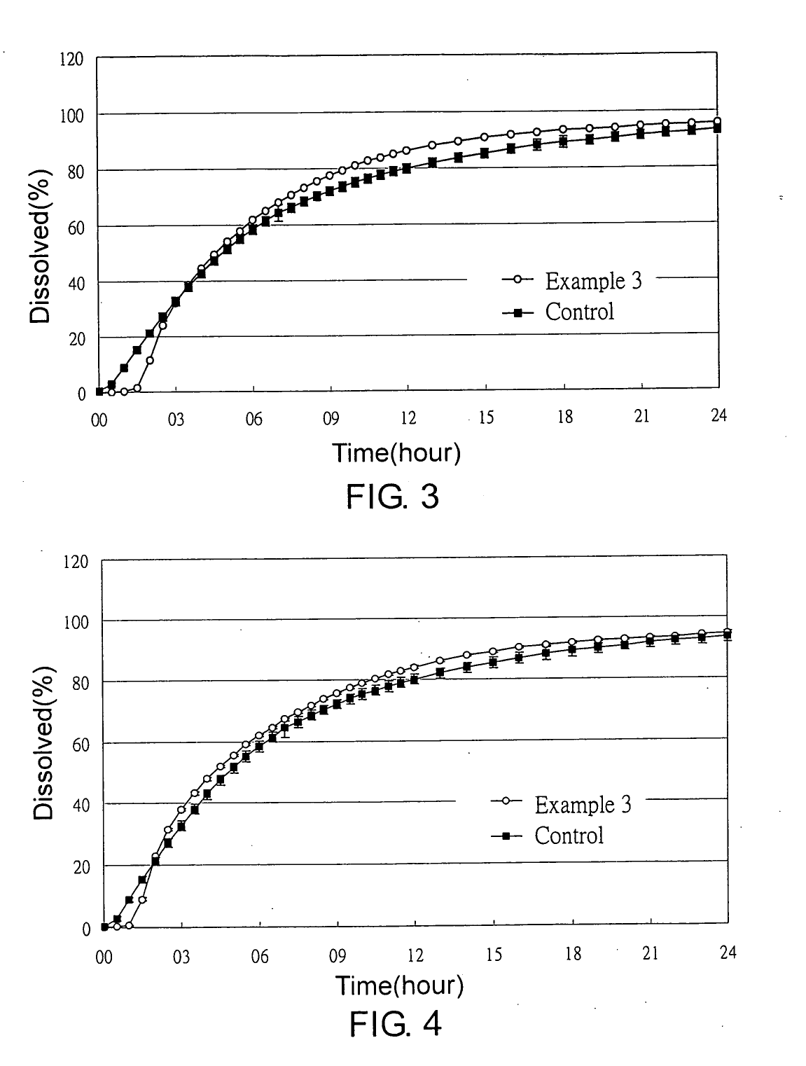

With reference to FIGS. 2 to 4, results show that the dissolution rate of the sustained release formulation containing venlafaxine or a pharmaceutically acceptable salt of venlafaxine respectively obtained from Examples 2 to 4 was similar as control.

Those persons skilled in the art will recognize various modifications and variations of the present invention without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

What is claimed is:

1. An oral sustained release formulation comprising
a core,
a medicinal layer containing vanlafaxine or a pharmaceutically acceptable salt of venlafaxine, with which the core is coated around, and
a release-modulating layer containing a release-modulating agent, with which the medicinal layer is coated around.

2. The oral sustained release formulation as claimed in claim 1, wherein the core is selected from a group consisting of sugar granule, sugar-starch granule and microcrystal cellulose.

3. The oral sustained release formulation as claimed in claim 1, wherein the core is made by one or more excipients.

4. The oral sustained release formulation as claimed in claim 3, wherein the excipient is one or more selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

5. The oral sustained release formulation as claimed in claim 1, wherein the medicinal layer comprises about 20% to 35% (w/w) venlafaxine or venlafaxine hydrochloride.

6. The oral sustained release formulation as claimed in claim 1, the medicinal layer further comprises a binder, a plastic agent, an anti-binding agent and/or a diluent.

7. The oral sustained release formulation as claimed in claim 6, wherein the binder in the medicinal layer is one or more selected from the group of polyvinyl pyrrolidone (PVP), hydroxypropyl cellulose (HPC), hydroxypropyl methylcellulose (HPMC), vinyl acetate (VA), polyvinyl alcohol (PVA), methylcellulose (MC), ethyl cellulose (EC), hydroxypropyl methylcellulose phthalate (HPMCP), cellulose acetate propionate (CAP), xanthan gum, alginic acid, alginate, methacrylic acid-methacrylate copolymer, methacrylic acid-methyl methacrylate copolymer and polyvinyl acetate phthalate (PVAP).

8. The oral sustained release formulation as claimed in claim 6, wherein the plastic agent in the medicinal layer is one or more selected from the group consisting of glycerol, polyethylene glycol, triethyl citrate, tributyl citrate, 3-propyl acetate, diethyl phthalate and dibutyl phthalate.

9. The oral sustained release formulation as claimed in claim 6, wherein the diluent in the medicinal layer is one or more selected from the group consisting of lactose, starch, mannitol, sodium hydroxylpropyl, glycol sodium starch, sodium chloride, potassium chloride, pigment, alginate, talcum powder, titanium dioxide, stearic acid, stearic salt, microcrystalline cellulose, glycerol, polyethylene glycol, triethyl citrate, tributyl citrate, 3-propyl acetate, calcium monophosphate, trisodium phosphate, calcium sulfate, cyclodextrin and castor oil.

10. The oral sustained release formulation as claimed in claim 1, wherein the release-modulating agent in the release-modulating layer is one or more selected from the group consisting of polyvinyl pyrrolidone (PVP), HPMC, EC, methacrylic acid-methacrylate copolymer and methacrylic acid-methyl methacrylate copolymer.

11. The oral sustained release formulation as claimed in claim 1, wherein the release-modulating layer further comprises an anti-binding agent and/or a plastic agent.

12. The oral sustained release formulation as claimed in claim 11, wherein the anti-binding agent in the release-modulating layer is one or more selected from the group consisting of talcum powder, titanium dioxide, stearic acid, stearic salt, sodium stearyl fumarate, glyceryl behenate, kaolin and aerosol.

13. The oral sustained release formulation as claimed in claim 11, wherein the plastic agent in the release-modulating layer is one or more selected from the group consisting of glycerol, polyethylene glycol, triethyl citrate, tributyl citrate, 3-propyl acetate, diethyl phthalate and dibutyl phthalate.

14. The oral sustained release formulation as claimed in claim 1, which comprises about 30% to 40% (w/w) the core, about 20% to about 35% (w/w) venlafaxine or venlafaxine hydrochloride and about 5% to about 20% (w/w) release-modulating layer.

15. The oral sustained release formulation as claimed in claim 14, which comprises about 35% (w/w) the core, about 30% (w/w) venlafaxine or venlafaxine hydrochloride and about 10% to about 16% (w/w) release-modulating layer.

16. A method for preparing a sustained release formulation, comprising
(a) providing a core,
(b) coating one or more medicinal layers on the core to obtain a granule, wherein the medicinal layer containing venlafaxine or a pharmaceutically acceptable salt of venlafaxine,
(c) coating one or more release-modulating layer on the granule, wherein the release-modulating layer containing a release-modulating agent, and
optionally (d) repeating step (b) and step (c).

17. The method as claimed in claim 16, which further comprises repeating (b) and (c) once or more.

18. The method as claimed in claim 18, wherein the core is provided by wet granulation.

19. The method as claimed in claim 16, wherein the core is of a diameter of from 0.5 to 0.85 mm.

20. A pharmaceutical composition comprising a sustained release formulation prepared according to the method as claimed in claim 16.
